(19) United States
(12) Patent Application Publication
(10) Pub. No.: US 2025/0332003 A1
(43) Pub. Date: Oct. 30, 2025

(54) TORQUE SENSING AND DETERMINATION FOR A PROSTHETIC JOINT ACTUATION SYSTEM

(71) Applicant: Össur Iceland ehf, Reykjavík (IS)

(72) Inventors: Guðni Ingimarsson, Reykjavik (IS); David Landry, St-Jean-Chrysostôme (CA); Hildur Inga Þorsteinsdóttir, Reykjavik (IS); Ragnar Sverrisson, Reykjavik (IS); Andri Bjorn Eidsson, Reykjavik (IS)

(21) Appl. No.: 19/189,914

(22) Filed: Apr. 25, 2025

Related U.S. Application Data
(60) Provisional application No. 63/640,758, filed on Apr. 30, 2024.

(51) Int. Cl. A61F 2/64; A61F 2/70; A61F 2/76

(52) U.S. Cl. CPC A61F 2/64; A61F 2002/701; A61F 2002/7645

(57) ABSTRACT

A prosthetic joint device may include a base coupled to an actuator of the joint mechanism. The device may include a first arm and a second arm extending from the base with a gap between the first arm and the second arm. The first arm and the second arm may form a closed loop. Distal ends of the first arm and the second arm may be coupled together at a distal connection point. A distal attachment portion including an opening may be rotatably coupled to a shank portion of the prosthetic device. The device may include a sensor to measure rotation of the distal attachment portion relative to the shank portion when the torque is applied to the base.

### INCORPORATION BY REFERENCE TO ANY PRIORITY APPLICATIONS

This application claims priority to U.S. Provisional Application No. 63/640,758, filed Apr. 30, 2024, which is incorporated herein by reference. Any and all applications for which a foreign or domestic priority claim is identified in the Application Data Sheet as filed with the present application are hereby incorporated by reference under 37 CFR 1.57.

### BACKGROUND

### Field

The present disclosure relates to a prosthetic joint, and more particularly, aspects of the present disclosure relate to determining a torque applied to an actuator of the prosthetic joint by an external force.

### Description of the Related Art

A few types of joint actuation mechanisms for prosthetic devices are known in the art. Usually, joint actuation mechanisms form part of a prosthetic device and include a housing for an actuator. The actuator can include a motor and a shaft in communication with a reducer, which communicates with an output to cause the joint to rotate about an axis thereof. Actuation mechanisms can provide measurement of the torque applied to the joint by an external force, which can be either from a prosthetic user or the motor of the actuator.

### SUMMARY

For purposes of this summary, certain aspects, advantages, and novel features are described herein. It is to be understood that not necessarily all such advantages may be achieved in accordance with any particular aspect. Thus, for example, those skilled in the art will recognize the disclosures herein may be carried out in a manner that achieves one or more advantages taught herein without necessarily achieving other advantages as may be taught or suggested herein.

In some aspects, a joint mechanism with a torque sensing transmission assembly may increase the stability of the joint mechanism during use. In some aspects, the torque sensing transmission assembly may reduce a weight of the joint mechanism and/or the prosthetic device. In some aspects, the torque sensing transmission assembly may increase comfort of the prosthetic device. In some aspects, the torque sensing transmission assembly may allow the prosthetic device to control all rotational movement of the joint mechanism. In some aspects, the torque sensing transmission assembly may allow rotational movement of the joint mechanism to be a controlled electromechanical movement.

In some aspects, a transmission assembly for a joint mechanism of a prosthetic device, wherein a torque applied to the joint mechanism is used to control a rotational movement of the joint mechanism, may include a base coupled to an actuator of the joint mechanism; a first arm extending from the base to a distal end of the first arm, the first arm being a rigid arm, and the distal end of the first arm may be coupled to a shank structure of the prosthetic device so that the first arm may be configured to rotate the shank structure about the base when a torque is applied to the base, the first arm may deflect by a first amount when the torque is applied to the base; a second arm extending from the base to a distal end of the second arm, the second arm being a rigid arm, a proximal end of the second arm may be coupled to a proximal end of the first arm so that when the torque is applied to the base, the second arm may deflect to a second amount that is greater than the first amount; and a sensor configured to determine a movement of the distal end of the second arm when the torque is applied to the base.

In some aspects, the first arm and the second arm may extend distally from the base at an angle from a longitudinal axis of the shank structure.

In some aspects, the second arm may be positioned forward of the first arm when in use.

In some aspects, the distal end of the second arm may be a free end and the sensor may be a linear sensor.

In some aspects, the transmission assembly may further include a magnet coupled to the distal end of the second arm, and the sensor may include a hall effect sensor.

In some aspects, the sensor may include a pin coupled to the shank structure, and the distal end of the second arm may be coupled to the pin so the distal end of the second arm may rotate the pin when the torque is applied to the base.

In some aspects, the first arm may be curved along at least a portion of the first arm, the second arm may be curved along at least a portion of the second arm, and the sensor may be a rotation sensor.

In some aspects, the transmission assembly may further include a gap between the first arm and the second arm.

In some aspects, a proximal portion of the first arm may be wider than a proximal portion of the second arm.

In some aspects, the first arm and the second arm may include titanium or aluminum.

In some aspects, the prosthetic device may include a prosthetic knee device.

In some aspects, the first arm may be curved along at least a portion of the first arm.

In some aspects, the first arm may include a proximal portion, a distal portion, and a curved intermediate portion between the proximal portion and the distal portion.

In some aspects, the first arm may be tapered along at least a portion of a length of the first arm.

In some aspects, a distal portion of the first arm may extend perpendicular to a longitudinal axis of the shank structure.

In some aspects, the second arm may be tapered along at least a portion of its length.

In some aspects, a prosthetic knee device may include a joint mechanism positioned between a shank structure and an adjacent prosthetic portion or a limb segment of a user, the joint mechanism may include an actuator; a transmission assembly, wherein a torque applied to the joint mechanism is used to control a rotational movement of the joint mechanism, the transmission assembly may include: a base coupled to the actuator of the joint mechanism; a first arm extending from the base to a distal end of the first arm, the first arm being rigid, the distal end of the first arm may be coupled to a shank structure of the prosthetic knee device so that the first arm may be configured to rotate the shank structure about the base when a torque is applied to the base, the first arm may deflect by a first amount when the torque is applied to the base; a second arm extending from the base to a distal end of the second arm, the second arm being a rigid arm, a proximal end of the second arm may be coupled to a proximal end of the first arm so that when a torque is applied to the base, the second arm may deflect to a second around that is greater than the first amount; and a sensor configured to determine a movement of the distal end of the second arm when the torque is applied to the base.

In some aspects, the movement of the distal end of the second arm may be used to determine the torque applied to the base.

In some aspects, the movement of the distal end of the second arm may be used as an input to control the actuator.

In some aspects, the transmission assembly may further include a magnet coupled to the distal end of the second arm, and the sensor may include a hall effects sensor.

In some aspects, the sensor may include a pin coupled to the shank structure, the distal end of the second arm may be coupled to the pin so the distal end of the second arm may rotate the pin when the torque is applied to the base.

In some aspects, a transmission assembly for a joint mechanism of a prosthetic device, wherein a torque applied to the joint mechanism is used to control a rotation movement of the joint mechanism, may include an output component including: a base coupled to an actuator of the joint mechanism; a first arm and a second arm forming a closed loop; the first arm may extend from the base to a distal end of the first arm at a distal end of the output component; the second arm may extend from the base to a distal end of the second arm at the distal end of the output component, the distal end of the second arm and the distal end of the first arm may be coupled together at a distal connection point positioned at the distal end of the output component; a gap between the first arm and the second arm may extending from the base to the distal connection point; a distal attachment portion including an opening and rotatably coupled to a shank portion of the prosthetic device, the distal attachment portion including an opening; and a sensor configured to measure rotation of the distal attachment portion relative to the shank portion of the prosthetic device when a torque is applied to the base.

In some aspects, the distal attachment portion extends proximally from the distal connection portion and is positioned inside the gap.

In some aspects, the first arm and/or the second arm may include a proximal portion, a distal portion, and an intermediate portion, the proximal portion may extend distally from the base, and the intermediate portion may extend between the proximal portion and the distal portion.

In some aspects, the intermediate portion may be curved outward away from a longitudinal axis of the output component extending from a proximal end of the output component to the distal end of the output component.

In some aspects, the distal portion may extend inward from the intermediate portion to the distal connection point.

In some aspects, the intermediate portion of each of the first arm and the second arm may be tapered along a length of the intermediate portion.

In some aspects, a width of the gap may increase a long a length of the intermediate portion of the first arm and the second arm.

In some aspects, the output component may be bent at a connection between the proximal portion and the intermediate portion so a first portion of the intermediate portion extends medially from the proximal portion at an angle.

In some aspects, the output component may be bent at a connection between the first portion of the intermediate portion and a second portion of the intermediate portion so the second portion and the distal portion extend parallel with the proximal portion.

In some aspects, the second portion of the intermediate portion and the distal portion may be medially offset from the proximal portion.

In some aspects, the first arm and the second arm may be flexible.

In some aspects, a fastener may be positioned in the opening of the distal attachment portion, the fastener may include a first fastener portion positioned in the opening of the distal attachment portion, a second fastener portion positioned over the first fastener portion on an outer side of the output component, and a third fastener positioned over the first fastener portion on an inner side of the output component.

In some aspects, the first fastener portion may be rotatably coupled to the second fastener portion and the third fastener portion, and the second fastener portion and the third fastener portion may be coupled to the shank portion of the prosthetic device.

In some aspects, the sensor may be positioned between the first fastener portion and the second fastener portion, and the sensor may be configured to measure rotation of the first fastener portion and the second fastener portion to measure the rotation of the distal attachment portion relative to the shank portion of the prosthetic device.

In some aspects, the prosthetic device may include a prosthetic knee device.

In some aspects, a prosthetic knee device may include a joint mechanism positioned between a shank structure and an adjacent prosthetic portion or a limb segment of a user, the joint mechanism may include an actuator; a transmission assembly, wherein a torque applied to the joint mechanism is used to control a rotational movement of the joint mechanism, the transmission assembly may include: an output component including: a base coupled to an actuator of the joint mechanism; a first arm and a second arm extending from the base and forming a closed loop at distal ends of the first and second arms; wherein the distal end of the second arm and the distal end of the first arm are coupled together at a distal connection point; a gap between the first arm and the second arm extending from the base to the distal connection point; a distal attachment portion including an opening and rotatably coupled to a shank portion of the prosthetic device; a fastener positioned in the opening of the distal attachment portion, the fastener configured to rotatably coupled the distal attachment portion to the shank portion of the prosthetic device; and a sensor configured to measure rotation of the distal attachment portion relative to the shank portion of the prosthetic device when a torque is applied to the base.

In some aspects, the rotation of the distal attachment portion relative to the shank portion may be used to determine the torque applied to the base or as an input to control the actuator.

In some aspects, the distal attachment point may extend proximally from the distal connection point and may be positioned in the gap.

In some aspects, the fastener may include a first fastener portion positioned in the opening of the distal attachment portion, a second fastener portion positioned over the first fastener portion on an outer side of the output component, and a third fastener positioned over the first fastener portion on an inner side of the output component, the first fastener portion may be rotatably coupled to the second fastener portion and the third fastener portion, and the second fastener portion and the third fastener portion may be coupled to the shank portion of the prosthetic device.

In some aspects, the sensor may be positioned between the first fastener portion and the second fastener portion, and the sensor may be configured to measure rotation of the first fastener portion and the second fastener portion to measure the rotation of the distal attachment portion relative to the shank portion of the prosthetic device.

### BRIEF DESCRIPTION OF THE DRAWINGS

These and other features, aspects, and advantages of the present disclosure are described with reference to the drawings of certain embodiments, which may not be to scale and are intended to schematically illustrate certain embodiments and not to limit the disclosure.

FIG. 1A illustrates a perspective view of an example of a prosthetic device.

FIG. 1B illustrates a perspective view of the example prosthetic device of FIG. 1A with a cover of the prosthetic device removed showing partially a joint actuation mechanism.

FIG. 2A illustrates a perspective view of an example joint mechanism of a prosthetic device.

FIG. 2B illustrates a perspective view of the example joint mechanism of FIG. 2A with an actuator housing removed.

FIG. 3A illustrates a side view of an example output component of a transmission assembly of a joint mechanism.

FIG. 3B illustrates a perspective view of a distal end of an example transmission assembly.

FIG. 3C illustrates a front view of the distal end of the example transmission assembly of FIG. 3B.

FIG. 4 illustrates a cross-section of the example prosthetic device of FIG. 1B showing the transmission assembly.

FIG. 5 illustrates a cross-section of an example prosthetic device with an example transmission assembly.

FIG. 6 illustrates an example output component of a transmission assembly coupled to a motor.

FIG. 7 illustrates an example motor of a joint mechanism with the actuator housing removed.

FIG. 8 illustrates an example block diagram of the prosthetic device disclosed herein.

FIG. 9 illustrates a perspective view of another example of a prosthetic device.

FIG. 10A illustrates a side view of an output component of a transmission assembly of the prosthetic device of FIG. 9.

FIG. 10B illustrates a perspective view of the transmission assembly of the prosthetic device of FIG. 9.

FIG. 10C illustrates a back view of the output component of FIG. 10A.

FIG. 10D illustrates a front view of the output component of FIG. 10A.

FIG. 11 illustrates a side view of another example output component of a transmission assembly.

FIG. 12A illustrates a side view of another example output component of a transmission assembly.

FIG. 12B illustrates a side view of another example output component of a transmission assembly.

### DETAILED DESCRIPTION

Although several aspects, examples, and illustrations are disclosed below, it will be understood by those of ordinary skill in the art that the system, methods, and devices described herein extend beyond the specifically disclosed aspects, examples, and illustrations and includes other uses of the system, methods, and devices and obvious modifications and equivalents thereof. The terminology used in the description presented herein is not intended to be interpreted in any limited or restrictive manner simply because it is being used in conjunction with a detailed description of certain specific aspects of the disclosure. In addition, aspects of the disclosure can comprise several novel features and no single feature is solely responsible for its desirable attributes or is essential to practicing the system, methods, and devices herein described.

The present disclosure provides example torque sensors for a prosthesis that can used in any load bearing application of a lower limb prosthesis, for example but not limited to a prosthetic knee joint which is part of a lower limb prosthesis. This invention allows for estimating the applied torque over the actuator of a prosthetic knee joint by an external force (prosthetic user or internal motor). The torque sensor disclosed herein can determine a torque applied to the knee joint by an external force (by the prosthetic user or the internal motor of the knee joint) as a control input for the prosthetic knee joint to smoothly control the actuator of the knee joint during prosthetic ambulation for all supported activities. The activities can include but are not limited to standing, walking, slopes, stair ascent and stair descent, sitting down, standing up, etc. A measured torque input into the control mechanism for a prosthetic knee joint can improve the smoothness of the control beyond what is possible with a simple position or velocity control. The torque estimation can improve impedance control of a prosthetic knee joint actuator compared to the control by a simple position or velocity control.

Torque estimation in actuators can be done using supply current to the actuator. However, such torque estimation requires the actuator to be completely locked by the supplied current for an accurate estimation. The torque sensor examples described herein can detect applied torque regardless of whether the actuator is locked for motion or not.

A prosthetic knee joint with a compliant transmission assembly, such as described in U.S. Pub. No. 20090299480A 1, the entirety of which is incorporated herein by reference, can provide measurement of the torque applied to the joint using a spring system of the compliant transmission assembly without locking the actuator. The actuator output shaft in such a knee joint can be connected to a spring system, which can then be connected to the knee frame. The rotation (and thereby the compression) of the spring system) can be measured at a lower pivot point. Based on the measured rotation, it is possible to estimate the torque applied to the knee joint. However, the spring system can create rotation of the knee joint when a user applies a torque to the knee joint, which may cause instability during use or at least result in undesirable or unwanted motion to the user. It can be beneficial to have a prosthetic knee joint that is more stable while still retaining the torque measurement performance of the compliant transmission assembly. The prosthetic devices disclosed herein can retain the torque sensing performance of a compliant transmission assembly, which can allow for the accuracy in the impedance control, while also providing greater comfort to the user as all rotational movement around the knee joint can be controllable by the device itself, resulting in a controlled electromechanical movement. Further, the design including the compliant transmission assembly may be more limited in the types of sensor (that is, a strain gauge) that can measure the compression of the spring system. The prosthetic device disclosed herein can allow a greater variety of sensors to be used for measuring the torque applied to the joint.

Another drawback of known joint actuation mechanisms is that they can be heavy and voluminous, directly affecting the weight and size of the device. The prosthetic devices disclosed herein can be lighter.

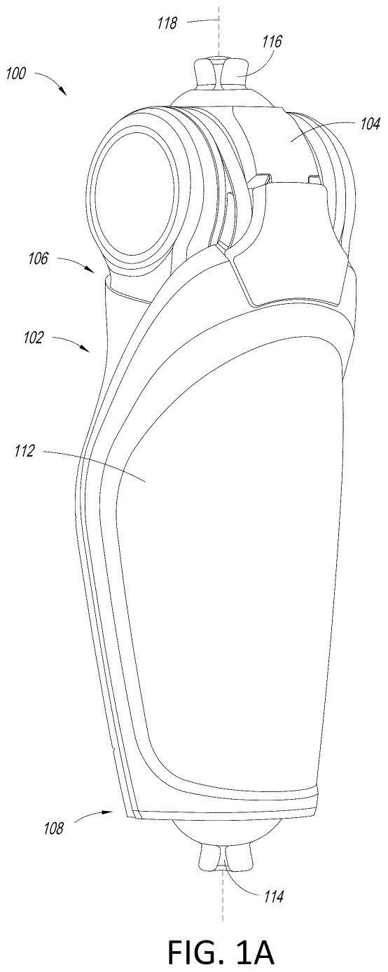

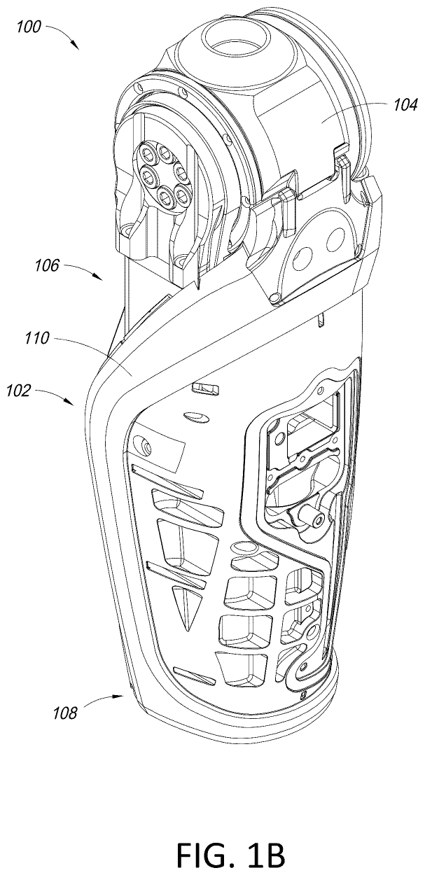

The prosthetic devices disclosed herein can address one or more of the disadvantages of existing joint actuation mechanisms discussed above, and/or other problems of the current prosthetic devices, and include one or more of the advantages disclosed herein, and/or other advantages. FIGS. 1A and 1B illustrate a prosthetic device 100. The prosthetic device 100 may include a shank portion 102 and a joint mechanism 104. The prosthetic device 100 may include a lower limb prosthetic. The prosthetic device 100 may include a prosthetic knee. Accordingly, the shank portion 102 may include a lower leg and the joint mechanism 104 may include a knee joint.

The shank portion 102 may extend between a proximal end 106 and a distal end 108. The shank portion 102 may include a shank structure 110 (shown in FIG. 1B), and a cover 112 (shown in FIG. 1A) positioned over the shank structure 110. The shank portion 102 may include a distal connector 114 at the distal end 108 of the shank portion 102. The distal connector 114 may couple the prosthetic device 100 to a prosthetic ankle and/or a prosthetic foot thereto. The joint mechanism 104 may be positioned at the proximal end 106 of the shank portion 102. The joint mechanism 104 may include a prosthetic connector 116. The prosthetic connector 116 may be connected to another adjacent prosthetic portion, such as a common socket, (not shown) that is mountable to a limb segment (e.g., leg stump) of a user. The joint mechanism 104 may be positioned between the shank portion 102 and the prosthetic portion and/or the limb (e.g., leg stump) of the user. The joint mechanism 104 may rotate the shank portion 102 relative to the prosthetic portion and/or the limb (e.g., leg stump) of the user.

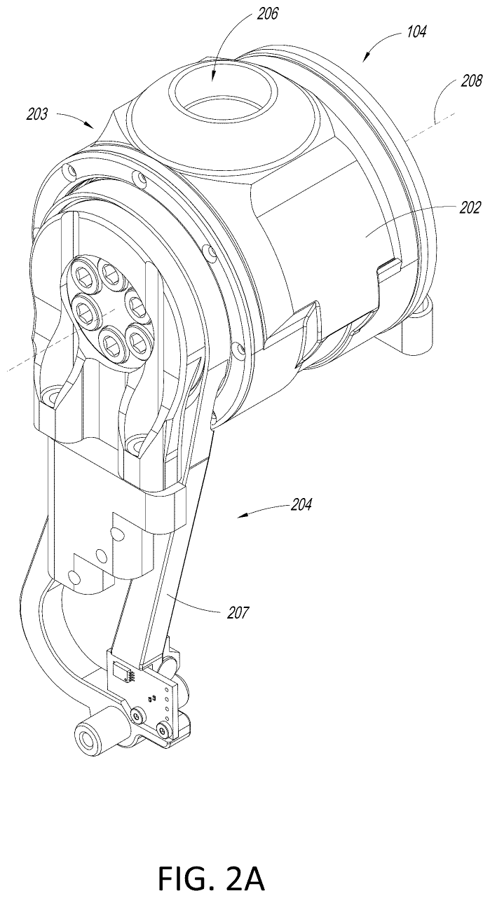

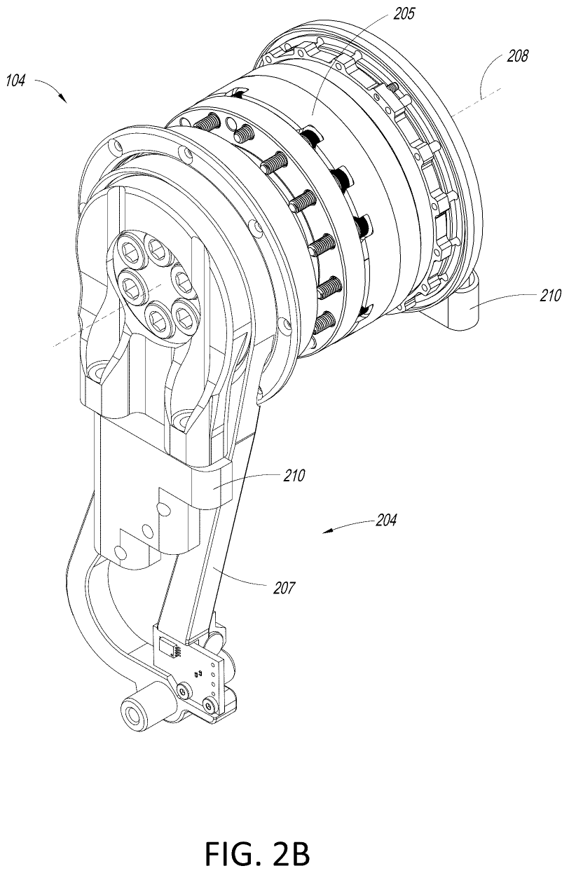

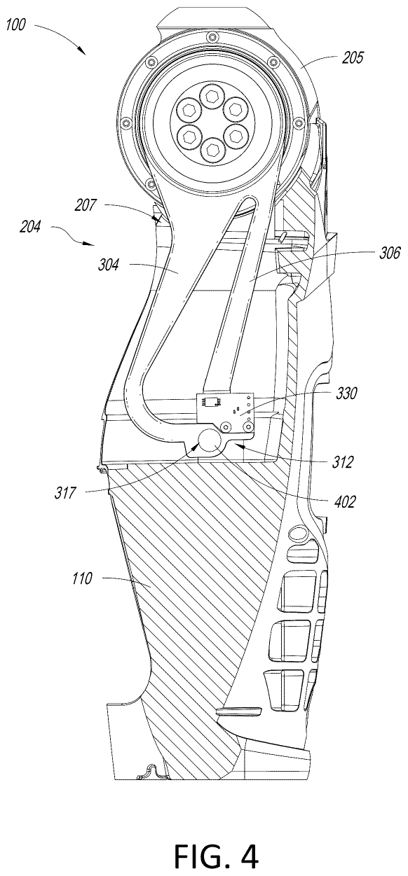

FIGS. 2A and 2B illustrate the joint mechanism 104. The joint mechanism 104 may include an actuator housing 202 and a transmission assembly 204. The actuator housing 202 may include a recess 206 at a proximal end 203 of the actuator housing 202. The prosthetic connector 116 (shown in FIG. 1A) may be coupled to or positioned in the recess 206. An actuator 205 (shown in FIG. 2B) may be positioned in the actuator housing 202. The actuator 205 may include a Brushless DC motor and/or any other type of actuator used for prosthetic devices. The transmission assembly 204 may be coupled to the actuator 205 at a proximal end of the transmission assembly 204. The actuator 205 may rotate portions of the transmission assembly 204 about a transverse axis 208 extending through the actuator 205. The transmission assembly 204 may be coupled to the shank structure 110 (shown in FIG. 4) at a distal end of the transmission assembly 204. When the actuator 205 rotates the portions of the transmission assembly 204 about the transverse axis 208, the actuator 205 may rotate the shank structure 110 around the transverse axis 208. Accordingly, the transmission assembly 204 includes an output component 207 transmitting rotation at the actuator 205 to the shank structure 110.

The joint mechanism 104 may include a support block 210. The support block 210 may be rotationally coupled to the actuator housing 202 and/or the actuator 205. The support block 210 may couple the actuator housing 202 and/or the actuator 205 to the shank structure 110 of the shank portion 102 without restricting the rotational movement of the shank structure 110 relative to the actuator 205. The support block 210 may prevent or inhibit the shank structure 110 from rotating relative to the joint mechanism 104 about a longitudinal axis 118 (shown in FIG. 1A). As shown in FIG. 1A, the longitudinal axis 118 may extend from the proximal end 106 of the shank portion 102 to the distal end 108 of the shank portion 102.

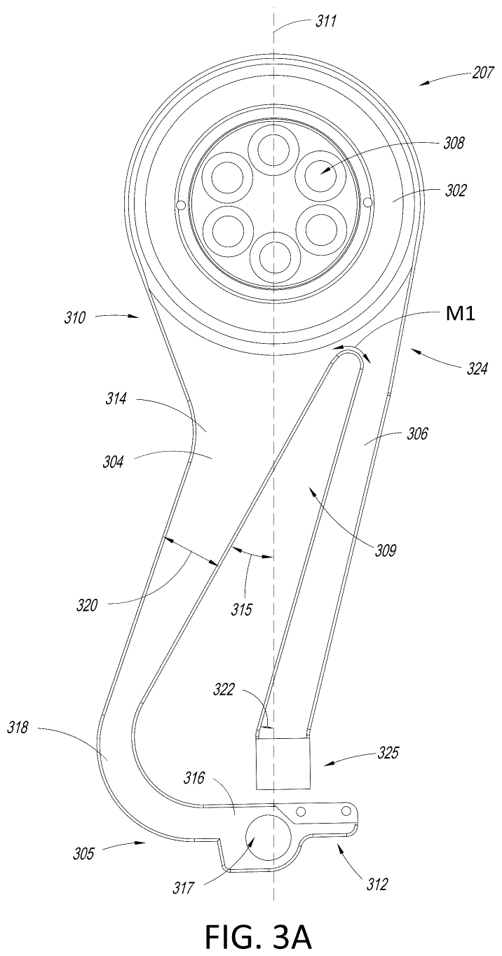

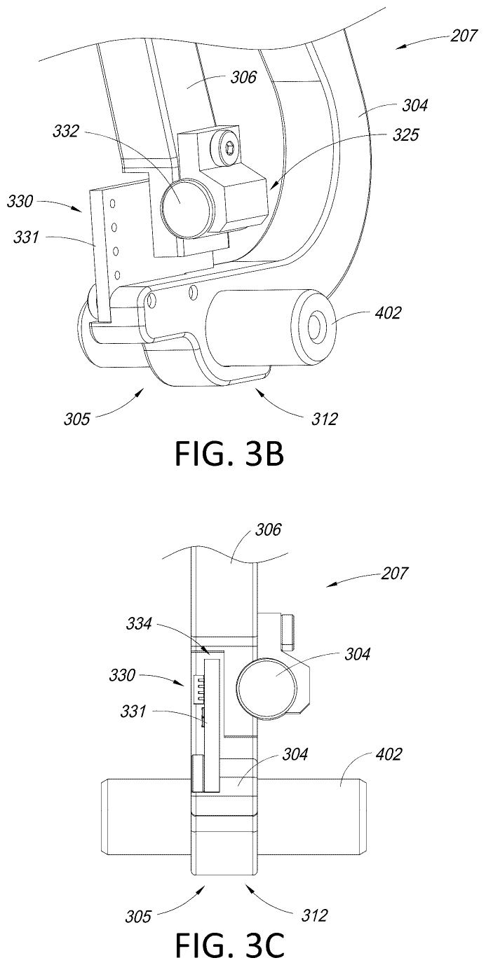

FIGS. 3A-3C illustrate the output component 207 of the transmission assembly 204. The output component 207 may include a base portion 302, a first arm 304, and/or a second arm 306. The base portion 302 may be coupled to the actuator 205 (shown in FIG. 2B). The base portion 302 may be coaxial with the actuator 205 along axis 208. Fasteners may be inserted through openings 308 in the base portion 302 in order to couple the base portion 302 to the actuator 205. The first arm 304 and/or the second arm 306 may extend from the base portion 302. The first arm 304 and/or the second arm 306 may extend from the base portion 302 in a generally distal direction (e.g., towards the distal end 108 of the shank portion 102). The first arm 304 and/or the second arm 306 are rigid. Throughout the present disclosure, a rigid part is at least more rigid and less elastic than a spring system. The rigid part can experience a small deflection under a load to an extent that is within the elastic zone of the Young's modulus of the material of the part. The small deflection does not result in a permanent deformation of the part.

The first arm 304 may extend between a proximal end 310 and a distal end 312. The first arm 304 may include a curve. Accordingly, the first arm 304 may be curved along a length of the first arm 304 between the proximal end 310 and the distal end 312. The distal end 312 of the first arm 304 may extend perpendicular to a longitudinal axis 311 extending from a proximal end 303 of the output component 207 to a distal end 305 of the output component 207.

The first arm 304 may include a proximal portion 314 and a distal portion 316. The proximal portion 314 may extend from the base portion 302 at the proximal end 310 in a generally distal direction (e.g., towards the distal end 108 of the shank portion 102). The proximal portion 314 may extend from the base portion 302 at an angle 315 with the longitudinal axis 311. The angle 315 may include an angle of about 15 degrees, about 20 degrees, about 25 degrees, about 26 degrees, about 27 degrees, about 28 degrees, about 29 degrees, about 30 degrees, about 31 degrees, about 32 degrees, about 33 degrees, about 34 degrees, about 35 degrees, about 40 degrees, about 45 degrees, about 50 degrees, about 55 degrees, about 60 degrees, and/or any value between the aforementioned values. The distal portion 316 of the first arm 304 may extend perpendicular to the longitudinal axis 311.

The first arm 304 may include an intermediate portion 318. The intermediate portion 318 may extend between the proximal portion 314 and the distal portion 316. The intermediate portion 318 may be curved along at least part of the intermediate portion 318.

The distal portion 316 may be coupled to the shank structure 110 of the shank portion 102, e.g., at or near the distal end 312, as described further below with reference to FIG. 4. Accordingly, the first arm 304 may be an output arm of the output component 207 and the first arm 304 may rotate the shank structure 110 when the actuator 205 rotates the output component 207. The distal portion 316 may include an opening 317 and a fastener 402 may be inserted through the opening 317 to couple the distal portion 316 to the shank structure 110 of the shank portion 102.

FIG. 4 illustrates a cross-section of the prosthetic device 100 with the transmission assembly 204. As described above, the distal end 312 of the first arm 304 of the output component 207 may be coupled to the shank structure 110. The fastener 402 (also shown in FIGS. 3B and 3C) may be inserted through the opening 317 in the distal portion 316 in order to couple the distal end 312 of the first arm 304 to the shank structure 110. Since the distal end 312 of the first arm 304 is fixed (e.g., coupled to the shank structure 110), the first arm 304 is an output lever arm that can transmit the torque at the joint (about axis 208 in FIGS. 2A-2B) to the shank structure 110. Since the distal end 312 of the first arm 304 is fixed (e.g., coupled to the shank structure 110) and the deflection of the first arm 304 is small, as will be described in more details elsewhere of the present disclosure, the torque applied to the joint mechanism 104 may be calculated based on the distance the distal end 325 of the second arm 306 moves relative to a sensor 330. The torque applied to the joint mechanism 104 may be used as an input for controlling the actuator 205 (shown in FIG. 2B).

With reference to FIG. 3A, the first arm 304 may include a width 320. The first arm 304 may be tapered along at least a portion of the first arm 304. The proximal portion 314 may be tapered along a length of the proximal portion 314. The width 320 of the first arm 304 may decrease (e.g., gradually decrease) along the proximal portion 314 from the base portion 302 to the intermediate portion 318 of the first arm 304. The width 320 of the first arm 304 may be larger at the proximal end 310 than at the opposite end of the proximal portion 314 where the proximal portion 314 transitions to the intermediate portion 318.

The second arm 306 may extend from the base portion 302 alongside the first arm 304. In use, the second arm 306 may be anterior or posterior relative to the first arm 304. The second arm 306 may extend from the base portion 302 at an angle 322. The angle 322 may include an angle of about 15 degrees, about 20 degrees, about 25 degrees, about 26 degrees, about 27 degrees, about 28 degrees, about 29 degrees, about 30 degrees, about 31 degrees, about 32 degrees, about 33 degrees, about 34 degrees, about 35 degrees, about 40 degrees, about 45 degrees, about 50 degrees, about 55 degrees, about 60 degrees, and/or any value between the aforementioned values. The angle 322 and the angle 315 may include a same angle. The angle 322 and the angle 315 may include different angles. The second arm 306 may extend parallel or at an angle to the proximal portion 314 of the first arm 304.

The second arm 306 may extend between a proximal end 324 and a distal end 325. The proximal end 324 of the second arm 306 may be coupled to the base portion 302. The distal end 325 may be a free end (e.g., unsupported end) of the second arm 306.

Accordingly, the second arm 306 may include a cantilever beam. The output component 207 of the transmission assembly 204 may include a gap 309 between the first arm 304 and the second arm 306.

The first arm 304 may be sized and/or shaped so the first arm 304 can rotate the shank structure 110 when the actuator 205 rotates the transmission assembly 204. An external force may be applied to the joint mechanism 104 by the prosthetic user or the actuator 205 to rotate the joint mechanism 104 about the axis 208 (see FIG. 2A), resulting in a torque applied to the first arm 304 and/or the shank structure 110. The torque applied to the first arm 304 and/or the shank structure 110 may be about 40 Nm to about 80 Nm, or about 50 Nm to about 70 Nm, or otherwise. The first arm 304 may be sized and/or shaped to reduce or minimize the amount of deflection of the first arm 304 when the torque is applied to the shank structure 110 and/or the first arm 304. A material of the output component 207, the first arm 304, and/or the second arm 306 may be selected so forces applied to the output component 207, the first arm 304, and/or the second arm 306 are within the elastic zone of the Young's modulus of the material during use of the prosthetic device 100. Accordingly, the output component 207, the first arm 304, and/or the second arm 306 does not permanently deform when forces are applied to the output component 207, the first arm 304, and/or the second arm 306. In some embodiments, the output component 207, the first arm 304, and/or the second arm 306 may include aluminum, titanium, and/or any other material with sufficient Young's modulus such that the output component 207, the first arm 304, and/or the second arm 306 results in a deflection and do not permanently deform during use. In some implementations, the second arm 306 can act as a cantilever beam by deflecting the distal end of the second arm 306 in the range of 0.00 mm and about 1.10 mm, or up to about 1 mm, or otherwise, without permanently deforming the second arm 306.

The second arm 306 and/or the gap 309 may be sized and/or shaped so the second arm 306 deflects to a greater extent than the deflection of the first arm 304 when the first arm 304 is strained due to the torque applied to the joint mechanism 104. The first arm 304 may be strained when a torque (resulting in a moment M 1) is applied to the shank structure 110 and/or the first arm 304. The first arm 304 may minimally deflect when the torque is applied to the shank structure 110 and/or the first arm 304. The proximal end 324 of the second arm 306 may be coupled (e.g., rigidly coupled) to the proximal end 310 of the first arm 304.

Accordingly, the first arm 304 may cause the second arm 306 to rotate by the moment M 1 when the torque is applied to the shank structure 110 and/or the first arm 304. The second arm 306 and/or the gap 309 may be sized and/or shaped so the second arm 306 amplifies the minimal deflection or deformation of the first arm 304. As disclosed elsewhere in the present disclosure, the distal end of the first arm 304 can be fixed such that the first arm 304 is slightly deformed when the torque is applied. The second arm 306 can rotate relative to the first arm 304 in the range of 0 degrees to about 0.7 degrees, or up to 0.6 degrees, or otherwise. The free distal end 325 of the second arm 306 can have a movement of a magnitude which amplifies the minimal amount of deformation of the first arm 304. In other words, the second arm 306 can deflect the same as a cantilever beam. The free distal end 325 of the second arm 306 can deflect a linear distance in the range of 0.00 mm to about 1.00 mm, or up to about 1 mm. This linear distance is greater than the amount of deformation in the first arm 304 and is therefore more easily measured by an appropriate sensor. The amplification of deflection in the second arm 306 can be due to one or more of the relative size (e.g., length, width, etc.) and shape of the first arm 304, the gap 309, and the second arm 306. The distal end 325 of the second arm 306 being a free end can make sure that the user may not experience the amplified movement of the second arm 306. The distal end 325 is not connected to any load-bearing parts of the prosthetic device 100.

As shown in FIGS. 3B and 3C, the transmission assembly 204 may include a sensor 330. The sensor 330 may be coupled to the distal end 312 of the first arm 304 and/or the distal portion 316 of the first arm 304. The second arm 306 may include a recess 334 at the distal end 325 of the second arm 306. The sensor 330 may extend into the recess 334 so the sensor 330 is aligned with the second arm 306. The sensor 330 may detect or determine a distance the distal end 325 of the second arm 306 moves when the second arm 306 deflects. The sensor 330 may include a linear displacement sensor or a linear position sensor that measures a linear component of the movement at the distal end 325 of the second arm 306. For example, the linear movement sensor may be a hall effect sensor that has a first component 331 that is coupled to the first arm 304, and a second, magnet component 332 coupled to the distal end 325 of the second arm 306. Accordingly, the first component 331 may determine the linear distance the distal end 325 of the second arm 306 moves based on a change in a magnetic field generated by the magnet component 332.

As the first arm 304 is now a rigid arm rather than a spring system, the motor in the actuator 205 can simulate a spring to provide spring damping control. In other words, the prosthetic device 100 and other devices disclosed herein can accurately measure the torque applied to the joint for control of the joint movement just as a prosthetic device with a compliant transmission assembly, and further improve user comfort as all rotational movement around the knee joint is controllable by the device itself. There is no unwanted rotational movement of the joint due to an external force applied to the joint. Rather, the rotational movement of the prosthetic device disclosed herein can be a controlled electromechanical movement.

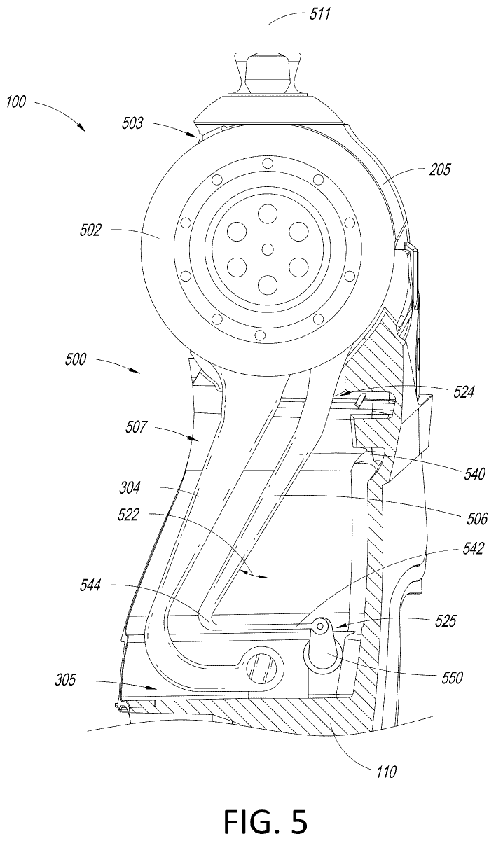

FIG. 5 illustrates a cross-section of the prosthetic device 100 with a transmission assembly 500. The transmission assembly 204 in FIGS. 2A-4 can have any of the features of the transmission assembly 500 in FIG. 5, and the transmission assembly 500 can have any features of the transmission assembly 204 except for the differences described with reference to FIG. 5. For example, the first arm 304 of the output component 507 of the transmission assembly 500 can be the same or substantially the same as the first arm 304 of the output component 207 of the transmission assembly 204. In FIG. 5, the second arm 506 of the output component 507 of the transmission assembly 500 may include a curved configuration rather than a straight arm 306 as shown in FIGS. 2A-4. The second arm 506 may extend between a proximal end 524 and a distal end 525. The second arm 506 may be curved along a length of the second arm 506 between the proximal end 524 and the distal end 525. The distal end 525 of the second arm 506 may extend perpendicular to a longitudinal axis 511 extending from a proximal end 503 of the transmission assembly 500 to a distal end 505 of the transmission assembly 500.

The second arm 506 may include a proximal portion 540 and a distal portion 542. The proximal portion 540 may extend from the base portion 502 of the output component 507 of the transmission assembly 500 is a generally distal direction. The proximal portion 540 may extend from the base portion 502 at an angle 522 with the longitudinal axis 511. The angle 522 may include an angle of about 15 degrees, about 20 degrees, about 25 degrees, about 26 degrees, about 27 degrees, about 28 degrees, about 29 degrees, about 30 degrees, about 31 degrees, about 32 degrees, about 33 degrees, about 34 degrees, about 35 degrees, about 40 degrees, about 45 degrees, about 50 degrees, about 55 degrees, about 60 degrees, and/or any value between the aforementioned values. The distal portion 542 of the second arm 506 may extend perpendicular to the longitudinal axis 511.

The second arm 506 may include an intermediate portion 544. The intermediate portion 544 may extend between the proximal portion 540 and the distal portion 542. The intermediate portion 544 may be curved along at least a portion of the intermediate portion 544.

The transmission assembly 500 may include a pin 550. The distal end 525 of the second arm 506 may be coupled (e.g., fixedly coupled) to the pin 550 at a first end of the pin 550. The pin 550 may be rotatably coupled to the shank structure 110 of the prosthetic device 100 at a second end of the pin 550 opposite the first end of the pin 550. The distal end 525 of the second arm 506 may be free to rotate relative to the shank structure 110 because of the rotational coupling between the second end of the pin 550 to the shank structure 110. In other words, the greater deflection of the distal end 525 of the second arm 506 is decoupled from any load-bearing components of the prosthetic device 100, similar to the cantilever beam design of the second arm 306 with a free distal end 325. When the second arm 506 deflects due to the torque applied to the joint system, the distal end 525 of the second arm 506 may rotate the pin 550 relative to the shank structure 110. The rotation of the pin 550 may be measured to determine the torque applied to the actuator 205 the joint system. The rotation of the pin 550 may be measured using a rotational sensor. The torque determined can be an input for controlling the actuator 205. The transmission assembly 500 uses the same principle of amplifying the displacement of the first arm 304 as the transmission assembly 204, but in the transmission assembly 500 the amplification is done using the different radius of the first arm 304 compared to the radius from the center of the actuator 205 to the second arm 506, which is a smaller pivot arm compared to the first arm 304.

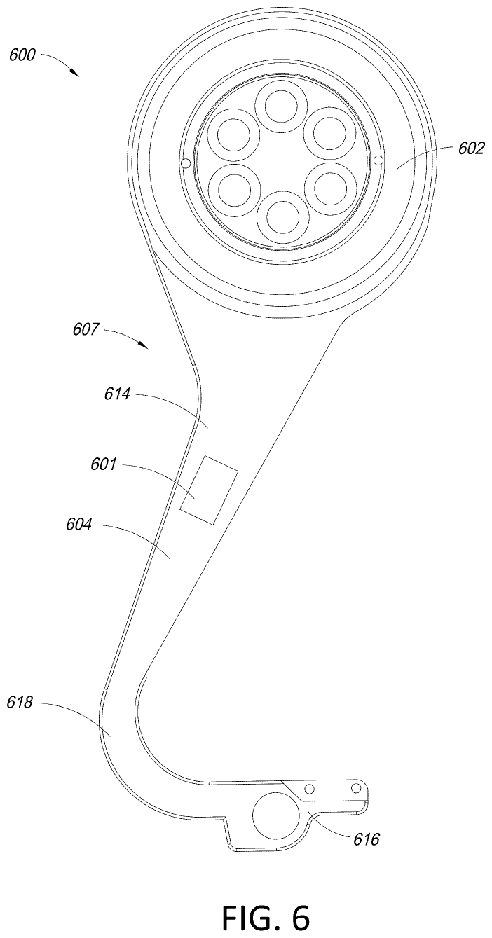

FIG. 6 illustrates a transmission assembly 600 with a strain gauge 601 on an output component 607. The transmission assemblies 204, 500 in FIGS. 2A-4 and 5 can have any features of the transmission assembly 600 in FIG. 6, and the transmission assembly 600 can have any features of the transmission assemblies 204, 500 except for the differences described with reference to FIG. 6. In FIG. 6, the output component 607 of the transmission assembly 600 may not include a second arm (such as the second arm 306 or the second arm 506 disclosed herein). The output component 607 of the transmission assembly 600 may include the base portion 602 and the arm 604. The arm 604 is the output lever arm. The arm 604 of the output component 607 of the transmission assembly 600 can be the same or substantially the same as the first arm 304 of the output component 207 of the transmission assembly 204. A strain gauge 601 may be coupled to the arm 604. The strain gauge 601 may be coupled to the proximal portion 614 of the arm 604, the intermediate portion 618 of the arm 604, and/or the distal portion 616 of the arm 604. The strain gauge 601 may measure or detect the strain in the arm 604 when a torque is applied to joint system, for example, on the shank structure 110 and/or the arm 604. The strain in the arm 604 may be used to by a controller in communication with the strain gauge 601 to determine the torque applied to an actuator of a joint system via the arm 604. The strain in the arm 604 may be used as an input by the controller for controlling the actuator.

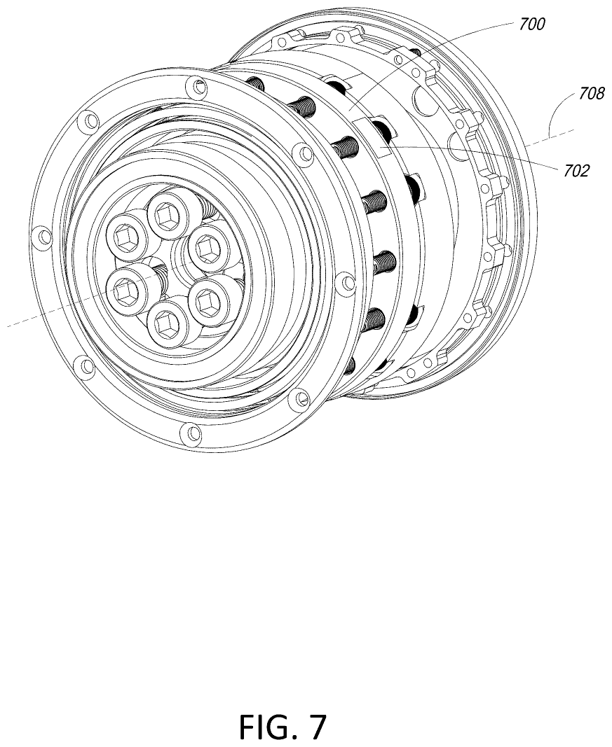

FIG. 7 illustrates an actuator 700 with a strain gauge 702. The actuator 205 in FIGS. 2A-2B can have any features of the actuator 700 of FIG. 7, and the actuator 700 can have any features of the actuator 205 except for the differences described with reference to FIG. 7. A transmission assembly of the prosthetic joint device implementing the actuator 700 may have any of the features of the transmission assembly examples disclosed elsewhere in the present disclosure and other transmission assembly examples disclosed herein may incorporate any of the features of the actuator 700. In FIG. 7, strain gauge 702 may be coupled to the actuator 700. The strain gauge 702 may measure or detect the strain in the actuator 700, for example, in an output lever (which may have any of the features of a first arm disclosed herein), when a torque is applied to the joint (about an axis 708) due to a force by the prosthetic user or a force applied by the actuator 700. The strain detected by the strain gauge 702 may be used by a controller in communication with the strain gauge 702 to determine the torque applied to the actuator 700. The strain detected by the strain gauge 702 may be used as an input by the controller for controlling the actuator.

It is to be appreciated that although the present application has been described with reference to the prosthetic device 100 being a lower limb prosthetic and/or a prosthetic knee, the prosthetic device 100 may include any prosthetic device with a joint mechanism.

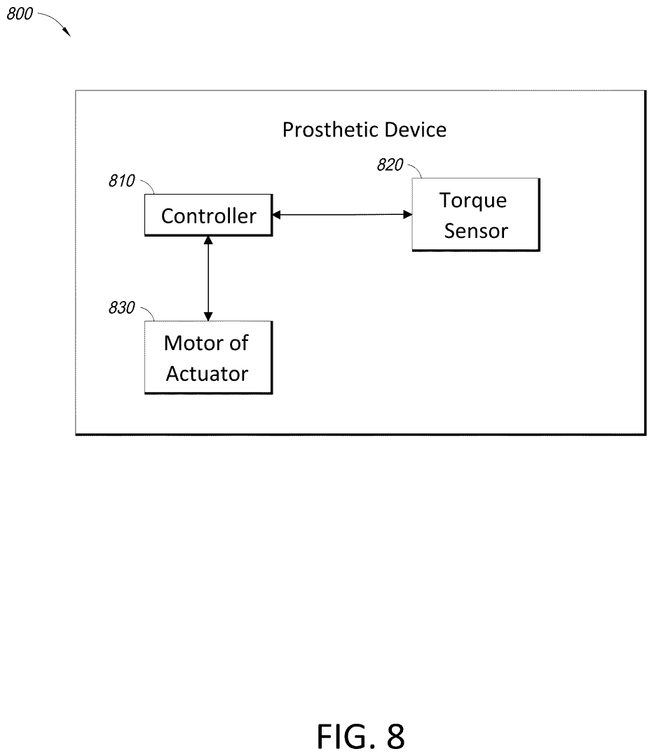

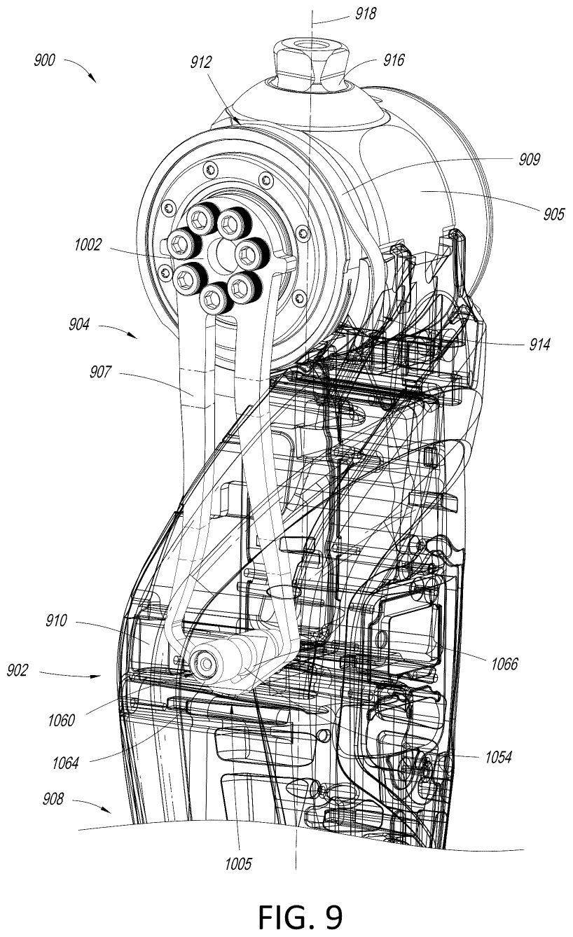

FIG. 8 illustrates a simplified block showing interaction between the controller 810, the torque sensor 820, and the motor 830 of the actuator of the prosthetic device 800. The device 800 shown in FIG. 8 can be any of the example devices disclosed herein and their variants. As described elsewhere in the present disclosure, the controller 810 can receive a torque measurement from the torque sensor 820. The torque sensor 820 can measure the strain in the output lever arm (e.g., the first arm in any of the example devices disclosed herein in FIGS. 1-7) and/or the rotation of the output component relative to the shank portion of the prosthetic device (e.g., rotation of the distal attachment portion in any of the example devices disclosed herein in FIG. 9-12B), either directly or indirectly as disclosed herein. The controller 810 can control the motor 830 of the actuator 840 at the joint to control the rotational movement of the joint system. As also described elsewhere in the present disclosure, the controller 810 can further simulate a spring system to provide damping control at the joint system.

FIG. 9 illustrates a prosthetic device 900. The prosthetic device 100 in FIGS. 1A-1B, 4 and 5 can have any of the features of the prosthetic device 900, and the prosthetic device 900 can have any features of the prosthetic device 100 except for the differences described with reference to FIG. 9. In FIG. 9, the prosthetic device 900 includes a transmission assembly 904 with an output component 907. The transmission assemblies 204, 500, 600 in FIGS. 2A-4, 5, and 6 can have any features of the transmission assembly 904 in FIG. 9, and the transmission assembly 904 can have any of the features of the transmission assemblies 204, 500, 600 except for the differences described with reference to FIG. 9. The transmission assembly 904 includes a securement member (e.g., a strap) 909. The securement member 909 may couple the actuator 905 to the shank structure 910. The securement member 909 may prevent or inhibit rotation of the shank structure 910 relative to the actuator 905 about the longitudinal axis 918 of the prosthetic device 900. The securement member 909 may extend between a first end 912 and a second end 914. The first end 912 may be coupled to the actuator 905. The second end 914 may be coupled to and/or extend into the shank structure 910. A bearing (not shown) may be positioned between the securement member 909 and the actuator 905 so the securement member 909 does not prevent or inhibit rotation of the actuator 905 about a transverse axis.

The transmission assembly 904 may be coupled to the actuator 905 at the proximal end of the transmission assembly 904 and coupled to the shank structure 910 of the prosthetic device 900 at the distal end of the transmission assembly 904. The coupling at the proximal end may be rotational/pivotal. The coupling at the distal end may be a fixed connection. The output component 907 of the transmission assembly 904 may be load bearing and may connect the actuator 905 to the shank structure 910 of the prosthetic device 900. Accordingly, the output component 907 may transfer rotation of the actuator 905 to the shank structure 910. Fasteners may be inserted into openings in a base portion 1002 of the output component 907 to couple the output component 907 to the actuator 905. A fastener 1060 at the distal end of the transmission assembly 904 may rotationally couple a distal end 1005 of the output component 907 to the shank structure 910.

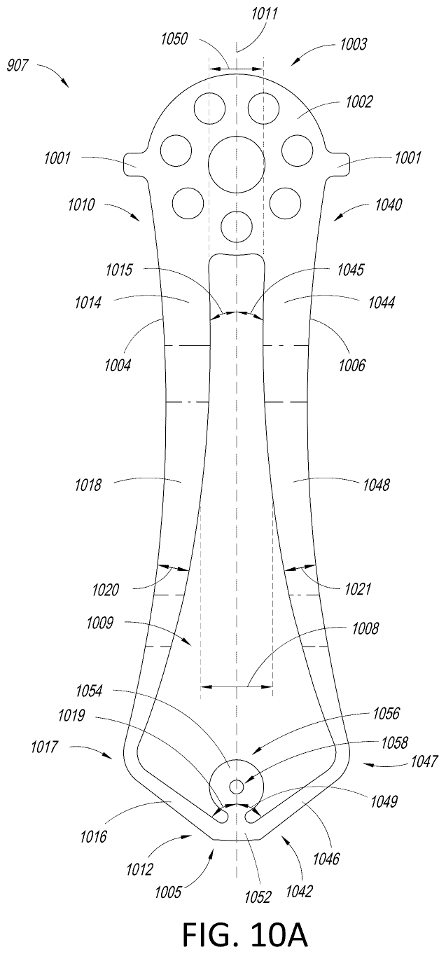

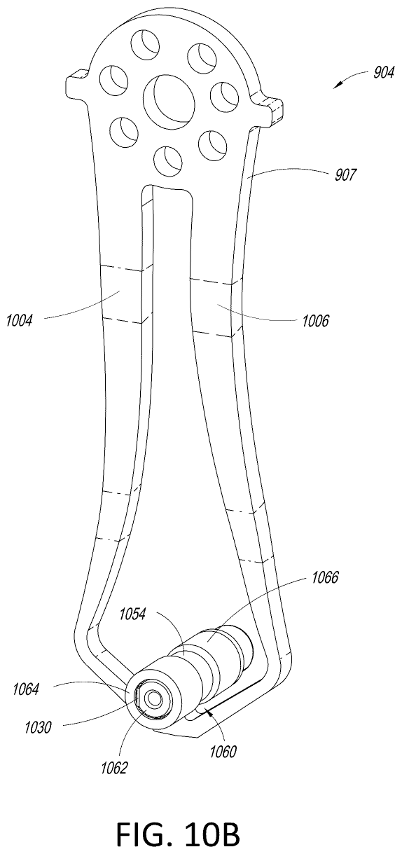

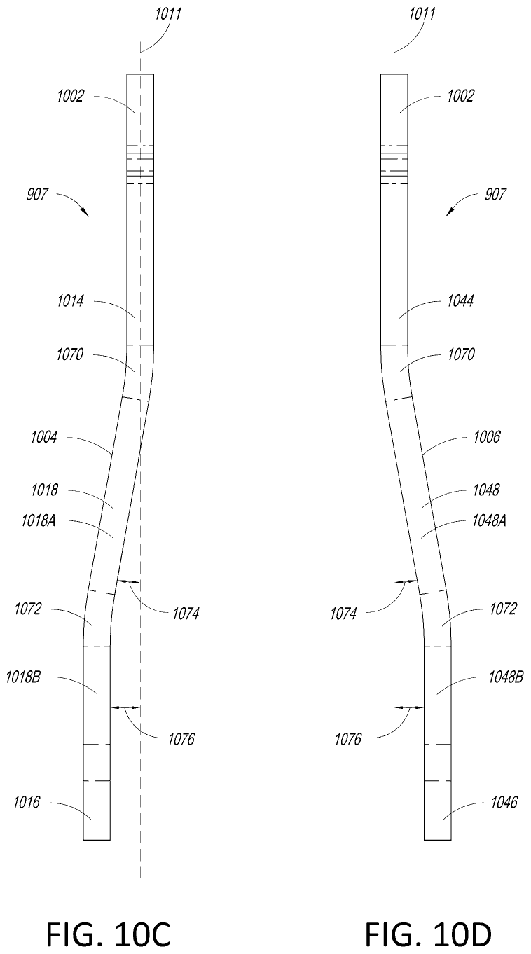

FIGS. 10A-10D illustrate the output component 907 of the transmission assembly 904. The output components 207, 507, 607 in FIGS. 2A-4, 5, and 6 can have any of the features of the output component 907, and the output component 907 can have any of the features of the output components 207, 507, 607, except for the differences described with reference to FIGS. 10A-10D. As shown in FIG. 10A, the output component 907 may include a base portion 1002, a first arm 1004, and/or a second arm 1006. The output component 907 may include protrusions 1001. The protrusions 1001 may extend from the base portion 1002 in a direction perpendicular to a longitudinal axis 1011 of the output component 907 extending from a proximal end 1003 of the output component 907 to a distal end 1005 of the output component 907. The protrusions 1001 may assist with manufacturing the output component 907. The protrusions 1001 may align the output component 907 in one or more machines that may shape, bend, or otherwise form the output component 907. One or more components of the prosthetic device 900 may be coupled to the protrusions 1001.

With continued reference to FIG. 10A, the first arm 1004 and/or the second arm 1006 may extend from the base portion 1002. The first arm 1004 and the second arm 1006 may extend from the base portion 1002 in a generally distal direction (e.g., toward a distal end 908 of the shank portion 902 shown in FIG. 9). The first arm 1004 and/or the second arm 1006 are flexible. Accordingly, when a force (e.g., a torque) is applied to the output component 907 (e.g., the first arm 1004 and/or the second arm 1006), the first arm 1004 and/or the second arm 1006 may deflect (e.g., flex). The deflection of the first arm 1004 and/or the second arm 1006 does not result in a permanent deformation. As will be described in greater detail elsewhere herein, the output component 907 may be shaped to maximize deflection the first arm 1004 and the second arm 1006 in the relatively small volume available in a prosthetic device. A distal attachment portion 1054 may be coupled to and positioned relative to the first arm 1004 and the second arm 1006 in order to promote or maximize rotation of the distal attachment portion 1054 when a torque is applied to the output component 907. The output component 907 may be stiffer than traditional spring systems, and may increase the stability of the prosthetic device 900.

The first arm 1004 may extend between a proximal end 1010 and a distal end 1012. The first arm 1004 may be curved along a length of the first arm 1004 between the proximal end 1010 and the distal end 1012. The first arm 1004 may curve outward (e.g., in a direction away from the longitudinal axis 1011 of the output component 907 and away from the second arm 1006) along part of the length of the first arm 1004. The first arm 1004 may curve inward (e.g., in a direction towards the longitudinal axis 1011 of the output component 907 and towards the second arm 1006) along another part of the length of the first arm 1004.

The first arm 1004 may include a proximal portion 1014, an intermediate portion 1018, and a distal portion 1016. The proximal portion 1014 may extend from the base portion 1002 at the proximal end 1010 in a generally distal direction (e.g., towards the distal end 908 of the shank portion 902). In some embodiments, the proximal portion 1014 may extend from the base portion 1002 in a direction parallel with the longitudinal axis 1011 of the output component 907. In some embodiments, the proximal portion 1014 may extend from the base portion 1002 in a direction inward towards the longitudinal axis 1011 of the output component 907. In these embodiment, the proximal portion 1014 may extend from the base portion 1002 at an angle 1015 with the longitudinal axis 1011. The angle 1015 may include an angle of about 1 degree, about 2 degrees, about 3 degrees, about 4 degrees, about 5 degrees, about 6 degrees, about 7 degrees, about 8 degrees, about 9 degrees, about 10 degrees, and/or any value between the aforementioned values. In some embodiments the angle 1015 may include an angle between 1 degree and 5 degrees.

The intermediate portion 1018 may extend from the proximal portion 1014 to the distal portion 1016. The intermediate portion 1018 may be curved along at least part of the intermediate portion 1018. The intermediate portion 1018 may be curved along an entire length of the intermediate portion 1018. The intermediate portion may curve outward (e.g., away from the longitudinal axis 1011 of the output component 907 and away from the second arm 1006) along the length of the intermediate portion 1018.

The distal portion 1016 may extend from the intermediate portion 1018 to the distal end 1012 of the first arm 1004. The distal portion 1016 may extend inward from the intermediate portion 1018 toward the longitudinal axis 1011 of the output component 907 and toward the second arm 1006. Accordingly, the first arm 1004 may be inward at a proximal end 1017 of the distal portion 1016 (e.g., the connection between the intermediate portion 1018 and the distal portion 1016). The distal portion 1016 may extend at an angle 1019 with the longitudinal axis 1011. The angle 1019 may include an angle of about 40 degrees, about 45 degrees, about 50 degrees, about 51 degrees, about 52 degrees, about 53 degrees, about 54 degrees, about 55 degrees, about 56 degrees, about 57 degrees, about 58 degrees, about 59 degrees, about 60 degrees, about 65 degrees, about 70 degrees, and/or any value between the aforementioned values. In some embodiments, the angle 1019 may include an angle between 45 degrees and 65 degrees. In some embodiments, the angle 1019 may include an angle between 50 degrees and 60 degrees. In some embodiments, the angle 1019 may include an angle between 53 degrees and 55 degrees.

Turning to the second arm 1006, the second arm 1006 may extend between a proximal end 1040 and a distal end 1042. The second arm 1006 may be curved along a length of the second arm 1006 between the proximal end 1010 and the distal end 1012. The second arm 1006 may curve outward (e.g., in a direction away from the longitudinal axis 1011 of the output component 907 and away from the first arm 1004). The second arm 1006 may curve inward (e.g., in a direction towards the longitudinal axis 1011 of the output component 907 and toward the first arm 1004) along another part of the length of the first arm 1004. in some embodiments, the second arm 1006 may be a mirror image of the first arm 1004 along the longitudinal axis 1011. In other embodiments, the second arm may look similar to the first arm 1004 but may not be identical in shape and/or dimensions.

The second arm 1006 may include a proximal portion 1044, and intermediate portion 1048, and a distal portion 1046. The proximal portion 1044 may extend from the base portion 1002 at the proximal end 1040 in a generally distal direction (e.g., towards the distal end 908 of the shank portion 902). In some embodiments, the proximal portion 1044 may extend from the base portion 1002 in a direction parallel with the longitudinal axis 1011 of the output component 907. In some embodiments, the proximal portion 1044 may extend from the base portion 1002 in a direction inward towards the longitudinal axis 1011 of the output component 907. In these embodiment, the proximal portion 1044 may extend from the base portion 1002 at an angle 1045 with the longitudinal axis 1011. The angle 1045 may include an angle of about 1 degree, about 2 degrees, about 3 degrees, about 4 degrees, about 5 degrees, about 6 degrees, about 7 degrees, about 8 degrees, about 9 degrees, about 10 degrees, and/or any value between the aforementioned values. In some embodiments the angle 1045 may include an angle between 1 degree and 5 degrees.

The intermediate portion 1048 may extend from the proximal portion 1044 to the distal portion 1046. The intermediate portion 1048 may be curved along at least part of the intermediate portion 1048. The intermediate portion 1048 may be curved along an entire length of the intermediate portion 1048. The intermediate portion may curve outward (e.g., away from the longitudinal axis 1011 of the output component 907 and away from the first arm 1004) along the length of the intermediate portion 1048.

The distal portion 1046 may extend from the intermediate portion 1048 to the distal end 1042 of the second arm 1006. The distal portion 1046 may extend inward from the intermediate portion 1048 toward the longitudinal axis 1011 of the output component 907 and toward the first arm 1004. Accordingly, the second arm 1006 may be curved inward at a proximal end 1047 of the distal portion 1046 (e.g., the connection between the intermediate portion 1048 and the distal portion 1046). The distal portion 1046 may extend at an angle 1049 with the longitudinal axis 1011. The angle 1049 may include an angle of about 40 degrees, about 45 degrees, about 50 degrees, about 51 degrees, about 52 degrees, about 53 degrees, about 54 degrees, about 55 degrees, about 56 degrees, about 57 degrees, about 58 degrees, about 59 degrees, about 60 degrees, about 65 degrees, about 70 degrees, and/or any value between the aforementioned values. In some embodiments, the angle 1049 may include an angle between 45 degrees and 65 degrees. In some embodiments, the angle 1049 may include an angle between 50 degrees and 60 degrees. In some embodiments, the angle 1049 may include an angle between 53 degrees and 55 degrees.

The distal portion 1016, 1046 of the first and second arms 1004, 1006 may be coupled together. The distal ends 1012, 1042 of the first and second arms 1004, 1006 may be coupled together at a distal connection point 1052. Accordingly, the first and second arms 1004, 1006 may form a closed loop-shaped arm extending from the base portion 1002. The distal connection point 1052 may be at the distal end 908 of the output component 907 and may be a distal most point of the output component 907. This is different from the configurations in, e.g., FIGS. 2A-5, where one arm is load-bearing and the other arm is non-loaded. The design with two distally connected arms as disclosed herein may include any of the advantages of the design with one load-bearing arm and one unloaded arm, as disclosed elsewhere herein. Additionally, the distally connected arms design may advantageously have a higher natural frequency than the one loaded arm/one unloaded arm design because the two arms are connected at the distal end of the output component 907 and work together as a closed structure, and/or because a width of the two arms decreases along a length of the arms. Additionally, the closed structure may advantageously reduce the vibration of the output component 907, which may increase the ability of sensor to accurately measure movement (e.g., rotation) of the output component 907 relative to prosthetic device, which in turn allows for more accurate torque estimation. Furthermore, in some embodiments, since the two arms are connected together, both arms are able to transfer rotation from the actuator 905 to the shank structure 910 of the prosthetic device 900, and/or both arms are able to store energy and restore energy, since energy is not lost due to movement of a free end of either of the arms.

The first arm 1004 and the second arm 1006 may be positioned on opposite sides of the longitudinal axis 1011 when viewed from an outer side of the output component 907, as shown in FIG. 10A. The output component 907 may include a gap 1009 between the first arm 1004 and the second arm 1006. The gap 1009 may extend along the length of the first arm 1004 and the second arm 1006 from the base 1002 to the distal connection point 1052. The proximal end 1010 of the first arm 1004 and the proximal end 1040 of the second arm 1006 may be separated by a distance 1050. The distance 1050 may be a width 1008 of the gap 1009 at the proximal ends 1010, 1040 of the first and second arms 1004, 1006. The width 1008 of the gap 1009 may increase along the length of the intermediate portions 1018, 1048 of the first and second arms 1004, 1006. The width 1008 of the gap 1009 may be largest at distal ends 1013, 1043 of the intermediate portion 1018, 1048. The width 1008 of the gap 1009 may decrease along the length of the distal portions 1016, 1046 of the first and second arms 1004, 1006.

The first arm 1004 may include a width 1020. The first arm 1004 may be tapered along at least a portion of the first arm 1004. The intermediate portion 1018 may be tapered along a length of the intermediate portion 1018. The width 1020 of the first arm 1004 may decrease (e.g., gradually decrease) along the intermediate portion 1018 from the proximal portion 1014 to the distal portion 1016. The width 1020 of the first arm 1004 may be larger at the proximal end 1010 than at the opposite end of the intermediate portion 1018 where the intermediate portion 1018 transitions to the distal portion 1016.

The second arm 1006 may include a width 1021. The second arm 1006 may be tapered along at least a portion of the second arm 1006. The intermediate portion 1048 may be tapered along a length of the intermediate portion 1048. The width 1021 of the second arm 1006 may decrease (e.g., gradually decrease) along the intermediate portion 1048 from the proximal portion 1044 to the distal portion 1046. The width 1021 of the second arm 1006 may be larger at the proximal end 1040 than at the opposite end of the intermediate portion 1048 where the intermediate portion 1048 transitions to the distal portion 1046.

The output component 907 may include a distal attachment portion 1054 extending from the distal connection point 1052. The distal attachment portion 1054 may extend in a generally proximal direction from the distal connection point 1052, so the distal attachment portion 1054 is positioned in the gap 1009 between the first and second arms 1004, 1006. Accordingly, when a force (e.g., a torque) is applied to the output component 907 (e.g., the first arm 1004 and/or the second arm 1006), and the first arm 1004 and/or the second arm 1006 deflect (e.g., flex), the distal connection point 1052 may rotate. In some embodiments, the distal attachment portion 1054 may be sized and shaped so a proximal end 1056 of the distal attachment portion 1054 does not extend proximal of the proximal ends 1017, 1047 of the distal portions 1016, 1046 of the first and second arms 1004, 1006. In some embodiments, the distal attachment portion 1054 may be sized and shaped so the proximal end 1056 of the distal attachment portion 1054 is aligned with the proximal ends 1017, 1047 of the distal portions 1016, 1046 of the first and second arms 1004, 1006.

The distal attachment portion 1054 may be rotatably coupled to the shank portion 902 of the prosthetic device 900, as shown in FIG. 9. The distal attachment portion 1054 may include an opening 1058. As shown in FIG. 10B, the distal attachment portion 1054 may receive a fastener 1060. The fastener 1060 may be positioned in the opening 1058 to couple the distal attachment portion 1054 to the shank portion 902 of the prosthetic device 900, as shown in FIG. 9. The fastener 1060 may include a first fastener portion 1062, a second fastener portion 1064, and a third fastener portion 1066. The first fastener portion 1062 may be positioned in opening 1058 of the distal attachment portion 1054. As shown in FIG. 9, The second fastener portion 1064 and the third fastener portion 1066 may be coupled to the shank portion 902 of the prosthetic device 900.

With reference to FIG. 10B, the second fastener portion 1064 and the third fastener portion 1066 may be positioned over the first fastener portion 1062 so the first fastener portion 1062 is positioned in the second fastener portion 1064 and the third fastener portion 1066. The second fastener portion 1064 may be positioned over a portion of the first fastener portion 1062 extending out of the distal attachment portion 1054 on the outer side of the output component 907. The third fastener portion 1064 may be positioned over a portion of the first fastener portion 1062 extending out of the distal attachment portion 1054 on an inner side of the output component 907 opposite the outer side of the output component 907.

The first fastener portion 1062 may be rotatably coupled to the second fastener portion 1064 and the third fastener portion 1066. Accordingly, when a force (e.g., a torque) is applied to the output component 907 (e.g., the first arm 1004 and/or the second arm 1006), and the distal connection point 1052 rotates, the first fastener portion 1062 may rotate relative to the second fastener portion 1064 and the third fastener portion 1066. The transmission assembly 904 may include a sensor 1030. The sensor 1030 may measure rotation of the first fastener portion 1062 relative to the second fastener portion 1064 and the third fastener portion 1066. The sensor 1030 may include a magnetic encoder and/or any other sensor suitable for measuring rotation. Since the second fastener portion 1064 and the third fastener portion 1066 are coupled the shank portion 902 (as shown in FIG. 9), the rotation of the first fastener portion 1062 relative to the second fastener portion 1064 and the third fastener portion 1066 may be the same as the rotation of the first fastener portion 1062 and the distal attachment portion 1054 relative to the shank portion 902. The measured rotation of the first fastener portion 1062 and the distal attachment portion 1054 may be used to determine the torque applied to the actuator 905 of the joint system. For example, the output component 907 (e.g., the first arm 1004, the second arm 1006, the distal attachment portion 1054, etc.) may be sized and/or shaped such that when a torque of 80 Nm is applied by the actuator 905 and the shank portion 902 rotates 3.5 degrees relative to the prosthetic connector 916 (shown in FIG. 9), the first fastener portion 1062 and the distal attachment portion 1054 rotate 3 degrees relative to the shank portion 902.

As shown in FIGS. 10C and 10D, the output component 907 may be bent. The first arm 1004 and the second arm 1006 may each include a first bend 1070 and a second bend 1072. The first bend 1070 may be positioned at a connection between the proximal portions 1014, 1044 and the intermediate portions 1018, 1048. The proximal portions 1014, 1044 may extend distally in a direction along the longitudinal axis 1011. At the first bend 1070, the first and second arms 1004, 1006 may be bent so first portions 1018A, 1048A of the intermediate portions 1018, 1048 extend medially from the proximal portions 1014, 1044 at an angle 1074 from the longitudinal axis 1011. The angle 1074 may include an angle of about 1 degree, about 2 degrees, about 3 degrees, about 4 degrees, about 5 degrees, about 6 degrees, about 7 degrees, about 8 degrees, about 9 degrees, about 10 degrees, about 11 degrees, about 12 degrees, about 13 degrees, about 14 degrees, about 15 degrees, about 16 degrees, about 17 degrees, about 18 degrees, about 19 degrees, about 20 degrees, and/or any value between the aforementioned values. In some embodiments, the angle 1074 may include an angle between 5 degrees and 15 degrees.

The first portions 1018A, 1048A may extend from the proximal portions 1014, 1044 to second portions 1018B, 1048B of the intermediate portions 1018, 1048. The second portions 1018B, 1048B may extend from the first portions 1018A, 1048B to the distal portions 1016, 1046. The second bend 1072 may be positioned at a connection between the first portions 1018A, 1048A and the second portions 1018B, 1048B. At the second bend 1072, the first and second arms 1004, 1006 may be bent so the second portions 1018B, 1048B of the intermediate portions 1018, 1048 extend from the first portions 1018A, 1048A of the intermediate portions 1018, 1048 parallel with the proximal portion 1014, 1044 (e.g., distally in a direction along the longitudinal axis 1011). The distal portions 1016, 1046 may extend from the intermediate portions 1018, 1048 parallel with the proximal portion 1014, 1044 (e.g., distally in a direction along the longitudinal axis 1011).

The second portions 1018B, 1048B of the intermediate portions 1018, 1048 and the distal portions 1016, 1046 may be offset medially a distance 1076 from the base 1002 and the proximal portions 1014, 1044 so the output component 907 is sized and/or shaped to fit in the prosthetic device 900. The distance 1076 may include a distance of about 0 mm, about 1 mm, about 2 mm, about 3 mm, about 4 mm, about 5 mm, about 6 mm, about 7 mm, about 8 mm, about 9, mm, about 10 mm, and/or any value between the aforementioned values.

In some embodiments, instead of having first and second bends 1070, 1072, the first and second arms 1004, 1006 may be curved along the length of the first and second arms 1004, 1006 so the distal portions 1016, 1046 are offset from the base 1002 and/or the proximal portion 1014, 1004.

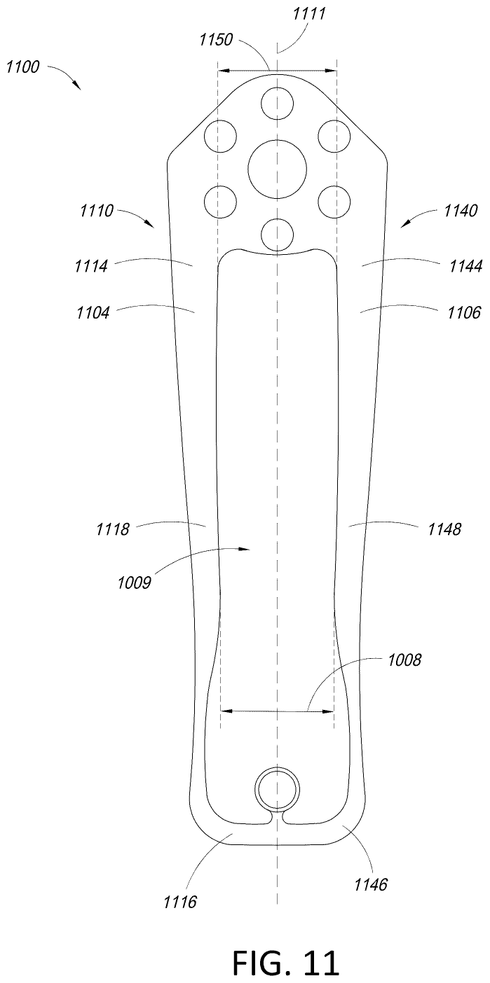

FIG. 11 illustrates another output component 1100 for a transmission assembly. The output components 207, 507, 607, 907 in FIGS. 2A-4, 5, 6, 9, and 10A-10D can have any of the features of the output component 1100, and the output component 1100 can have any of the features of the output components 207, 507, 607, 907 except for the differences described with reference to FIG. 11.

In FIG. 11, the distance 1150 between the first and second arms 1104, 1106 (e.g., the width 1108 of the gap 1109 at the proximal ends 1110, 1140 of the first and second arms 1104, 1106) may be larger than the distance 1050 between the between the first and second arms 1004, 1006 (e.g., the width 1008 of the gap 1009 at the proximal ends 1010, 1040 of the first and second arms 1004, 1006) of the output component 907. The proximal portions 1114, 1144 of the first and second arms 1104, 1106 and/or the intermediate portions 1118, 1148 of the first and second arms 1104, 1106 may be straight. Accordingly, the proximal portions 1114, 1144 of the first and second arms 1104, 1106 may not extend inward towards the longitudinal axis 1111 and/or the intermediate portions 1118, 1148 of the first and second arms 1104, 1106 may not curve outward (e.g., away from the longitudinal axis 1111) like the proximal portions 1014, 1044 and the intermediate portions 1018, 1048 of the first and second arms 1004, 1006 of the output component 907.

The distal portions 1116, 1146 of the first and second arms 1104, 1106 may extend inward from the intermediate portions 1118, 1148 in a direction perpendicular to the longitudinal axis 1111 of the output component 1100.

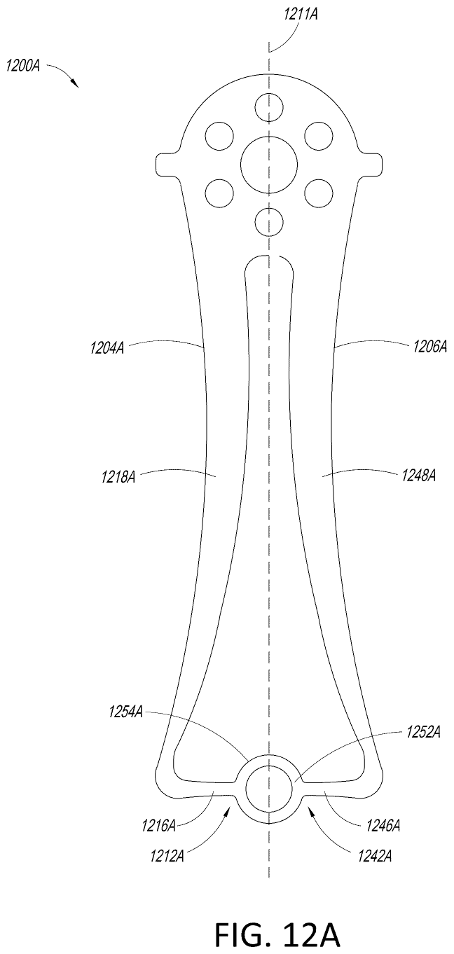

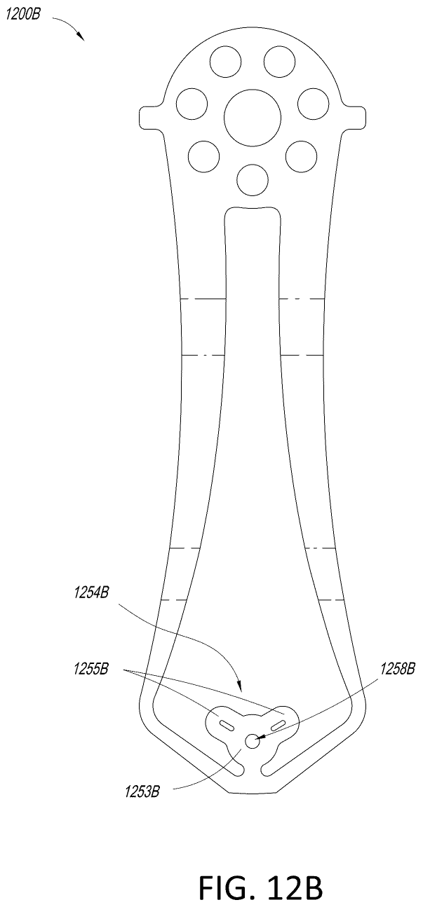

FIG. 12A illustrates another output component 1200A for a transmission assembly. The output components 207, 507, 607, 907, 1100 in FIGS. 2A-4, 5, 6, 9, 10A-10D, and 11 can have any of the features of the output component 1200A, and the output component 1200A can have any of the features of the output components 207, 507, 607, 907, 1100 except for the differences described with reference to FIG. 12A. In FIG. 12A, the distal portions 1216A, 1246A of the first and second arms 1204A, 1206A may extend inward from the intermediate portions 1218A, 1248A in a directions perpendicular to the longitudinal axis 1211A of the output component 1200A. The distal attachment portion 1254A may be positioned between the distal ends 1212A, 1242A of the first and second arms 1204A, 1206A, so the distal attachment portion 1254A forms the distal connection point 1252A, instead of extending proximally from the distal connection point 1052 like the distal attachment portion 1054 of the output component 907. Accordingly, the distal attachment portion 1254A may be aligned with the distal portions 1216A, 1246A of the first and second arms 1204A, 1206A.

FIG. 12B illustrates another output component 1200B for a transmission assembly. The output components 207, 507, 607, 907, 1100, 1200A in FIGS. 2A-4, 5, 6, 9, 10A-10D, 11, 12A can have any of the features of the output component 1200B, and the output component 1200B can have any of the features of the output components 207, 507, 607, 907, 1100, 1200A except for the differences described with reference to FIG. 12B. In FIG. 12B, the distal attachment portion 1254B of the output component 1200B may include a body 1253B and protrusions 1255B. The body 1253B may include the opening 1258B that may receive a fastener. The protrusions 1255B may extend out from the body 1253B. The protrusions 1255B may allow for compliance of the opening 1258B (e.g., may allow the opening 1258B to expand) in order to receive a fastener (e.g., fastener 1060). The protrusions 1255B may apply a force to the body 1253B to maintain a press fit connection between the body 1253B and the fastener to prevent or inhibit a portion of the fastener positioned in the opening 1258B from rotating relative to the body 1253B. The protrusions 1255B may reduce or minimize vibration of the body 1253B. A diameter of the 1258B may vary and may be larger or smaller than as shown in FIG. 12B without departing from the scope of this disclosure.

It is to be appreciated that although at least some of the output components are described herein as having a first arm and a second arm, the output components may instead include one (1) arm or three (3) arms, and/or any other number of arms without departing from the scope the present disclosure.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A transmission assembly for a joint mechanism of a prosthetic device, wherein a torque applied to the joint mechanism is used to control a rotation movement of the joint mechanism, the transmission assembly comprising:
an output component comprising:
a base coupled to an actuator of the joint mechanism;
a first arm and a second arm forming a closed loop;
wherein the first arm extends from the base to a distal end of the first arm at a distal end of the output component;
wherein the second arm extends from the base to a distal end of the second arm at the distal end of the output component, wherein the distal end of the second arm and the distal end of the first arm are coupled together at a distal connection point positioned at the distal end of the output component;
a gap between the first arm and the second arm extending from the base to the distal connection point;
a distal attachment portion comprising an opening and rotatably coupled to a shank portion of the prosthetic device, the distal attachment portion comprising an opening; and
a sensor configured to measure rotation of the distal attachment portion relative to the shank portion of the prosthetic device when a torque is applied to the base.

2. The transmission assembly of claim 1, wherein the distal attachment portion extends proximally from the distal connection portion and is positioned inside the gap.

3. The transmission assembly of claim 1, wherein the first arm and/or the second arm comprise a proximal portion, a distal portion, and an intermediate portion, the proximal portion extending distally from the base, and the intermediate portion extending between the proximal portion and the distal portion.

4. The transmission assembly of claim 3, wherein the intermediate portion is curved outward away from a longitudinal axis of the output component extending from a proximal end of the output component to the distal end of the output component.

5. The transmission assembly of claim 4, wherein the distal portion extends inward from the intermediate portion to the distal connection point.

6. The transmission assembly of claim 3, wherein the intermediate portion of each of the first arm and the second arm is tapered along a length of the intermediate portion.

7. The transmission assembly of claim 3, wherein a width of the gap increases a long a length of the intermediate portion of the first arm and the second arm.

8. The transmission assembly of claim 3, wherein the output component is bent at a connection between the proximal portion and the intermediate portion so a first portion of the intermediate portion extends medially from the proximal portion at an angle.

9. The transmission assembly of claim 8, wherein the output component is bent at a connection between the first portion of the intermediate portion and a second portion of the intermediate portion so the second portion and the distal portion extend parallel with the proximal portion.

10. The transmission assembly of claim 9, wherein the second portion of the intermediate portion and the distal portion are medially offset from the proximal portion.

11. The transmission assembly of claim 1, wherein the first arm and the second arm are flexible.

12. The transmission assembly of claim 1, wherein a fastener is positioned in the opening of the distal attachment portion, the fastener comprising a first fastener portion positioned in the opening of the distal attachment portion, a second fastener portion positioned over the first fastener portion on an outer side of the output component, and a third fastener positioned over the first fastener portion on an inner side of the output component.

13. The transmission assembly of claim 12, wherein the first fastener portion is rotatably coupled to the second fastener portion and the third fastener portion, and the second fastener portion and the third fastener portion are coupled to the shank portion of the prosthetic device.

14. The transmission assembly of claim 13, wherein the sensor is positioned between the first fastener portion and the second fastener portion, and wherein the sensor is configured to measure rotation of the first fastener portion and the second fastener portion to measure the rotation of the distal attachment portion relative to the shank portion of the prosthetic device.

15. The transmission assembly of claim 1, wherein the prosthetic device comprises a prosthetic knee device.

16. A prosthetic knee device comprising:
a joint mechanism positioned between a shank structure and an adjacent prosthetic portion or a limb segment of a user, the joint mechanism comprising an actuator;
a transmission assembly, wherein a torque applied to the joint mechanism is used to control a rotational movement of the joint mechanism, the transmission assembly comprising:
an output component comprising:
a base coupled to an actuator of the joint mechanism;
a first arm and a second arm extending from the base and forming a closed loop at distal ends of the first and second arms; wherein the distal end of the second arm and the distal end of the first arm are coupled together at a distal connection point;
a gap between the first arm and the second arm extending from the base to the distal connection point;
a distal attachment portion comprising an opening and rotatably coupled to a shank portion of the prosthetic device;
a fastener positioned in the opening of the distal attachment portion, the fastener configured to rotatably coupled the distal attachment portion to the shank portion of the prosthetic device; and
a sensor configured to measure rotation of the distal attachment portion relative to the shank portion of the prosthetic device when a torque is applied to the base.

17. The prosthetic knee device of claim 16, wherein the rotation of the distal attachment portion relative to the shank portion is used to determine the torque applied to the base or as an input to control the actuator.

18. The prosthetic knee device of claim 16, wherein the distal attachment point extends proximally from the distal connection point and is positioned in the gap.

19. The prosthetic knee device of claim 16, wherein the fastener comprises a first fastener portion positioned in the opening of the distal attachment portion, a second fastener portion positioned over the first fastener portion on an outer side of the output component, and a third fastener positioned over the first fastener portion on an inner side of the output component, wherein the first fastener portion is rotatably coupled to the second fastener portion and the third fastener portion, and the second fastener portion and the third fastener portion are coupled to the shank portion of the prosthetic device.

20. The prosthetic knee device of claim 19, wherein the sensor is positioned between the first fastener portion and the second fastener portion, and wherein the sensor is configured to measure rotation of the first fastener portion and the second fastener portion to measure the rotation of the distal attachment portion relative to the shank portion of the prosthetic device.
